(19) United States
(12) Patent Application Publication
(10) Pub. No.: US 2020/0163705 A1
(43) Pub. Date: May 28, 2020

(54) BONE SCREW ASSEMBLY AND METHOD FOR ANCHORING BONE PARTS

(71) Applicant: AD INNOVATIVE IMPLANTS LTD., Rishon LeZion (IL)

(72) Inventor: Amir Herman, Kiryat Ono (IL)

(73) Assignee: AD INNOVATIVE IMPLANTS LTD., Rishon LeZion (IL)

(21) Appl. No.: 16/618,819

(22) PCT Filed: Jun. 1, 2018
(86) PCT No.: PCT/IB2018/053940

§ 371 Date: Dec. 3, 2019

Related U.S. Application Data
(60) Provisional application No. 62/514,885, filed on Jun. 4, 2017.

(51) Int. Cl. A61B 17/86; A61B 17/80

(52) U.S. Cl. CPC A61B 17/8685; A61B 17/863; A61B 17/8605; A61B 17/8057

(57) ABSTRACT

Disclosed are bone screw assemblies and kits including the same. Also disclosed are methods for anchoring of bone parts using a bone screw, for example, in order to fix parts of a broken bone in a desired relative orientation. Embodiments of the bone screw assemblies include a screw with an enlarged screw head and a cap having an internal cavity in which the screw head is retained wherein the internal cavity and screw head are dimensioned to allow, during use, a limited movement of the screw head within the cavity including translation of the screw head in a direction parallel to the X-axis of the cap.

### RELATED APPLICATION

The present application gains priority from U.S. Provisional Patent Application No. U.S. 62/514,885 filed 4 Jun. 2017, which is included by reference as if fully set-forth herein.

### FIELD AND BACKGROUND OF THE INVENTION

The invention, in some embodiments, relates to the field of medicine, and more specifically, in some embodiments to bone screws and methods for anchoring bone parts, for example, in order to fix fragments of a broken bone in a desired relative orientation.

For the treatment of a fractured bone, it is known to fix two different bone fragments in a desired relative orientation with the use of a rigid plate spanning the fracture, the plate secured to the fragments by bone screws driven into the fragments.

Once the bone fragments are fixed in the desired relative orientation, the fracture can heal properly.

Such methods and suitable devices have been described in the art, see for example, US 2007/0233116, US 2011/0319942, US 2012/0143193 and US 2014/0330275.

### SUMMARY OF THE INVENTION

Some embodiments of the invention relate to bone screws and to methods for anchoring bone fragments using a bone screw, for example, in order to fix fragments of a broken bone in a desired relative orientation with an implant such as a bone fixation plate as known in the art of bone fracture repair.

In one aspect, the invention provides a bone screw assembly for securing a plate to a bone, comprising a screw having a shaft that defines an axis, a screw thread for engaging in the bone as the screw is rotated about the axis, an enlarged head at a proximal end of the shaft, and a cap fitting over the head of the screw, the cap having an external surface engageable in the plate and an inner cavity for receiving the head of the screw, wherein the head of the screw and the cavity in the cap are shaped and dimensioned in such a manner that, when in use, the screw head is capable of a limited sliding motion relative to the cap in a plane perpendicular to the shaft axis while being prevented from moving relative to the cap in the direction of the shaft axis.

In some embodiments, when in use for fixing a bone fixation plate (hereinfurther called “plate”) to fragments of a fractured bone, the screw head is slidable relative to the cap in only one direction within the orthogonal plane, the direction being transverse to the fracture plane of the bone to which the plate is secured. Thus, in the case where the plate is used in the repair of a fractured elongate limb, and the cap is fully locked in the plate, the screw head can move relative to the cap and the plate in a direction parallel to the axis of the limb.

Thus, according to an aspect of some embodiments of the invention there is provided a bone screw assembly for securing an implant (such as a plate as known in the art of bone fracture repair) to a bone comprising:
(i) a screw-shaft bearing a shaft screw-thread, the screw-shaft defining a screw-axis and having a distal tip and a proximal end, the screw-shaft, the shaft screw-thread and the distal tip configured to penetrate and engage the bone;
(ii) an enlarged screw head located at the proximal end of the screw-shaft;
(iii) a cap for engaging the implant having a top side, a bottom side, a width along an X-axis, a length along a Y-axis and a height along a Z-axis that passes through the top side and the bottom side, the cap having an inner surface defining a cavity in which the screw head is retained, a distal port being provided in the bottom side of the cap through which the screw-shaft passes, the distal port being dimensioned to prevent passage of the screw head therethrough;
   wherein the internal cavity, the distal port, the screw-shaft and the screw head are dimensioned to allow, during use, a limited movement of the screw head within the cavity, the limited movement including translation of the screw head in a direction parallel to the X-axis of the cap.

In some embodiments, the cap further comprises a cap screw thread on an outer surface thereof allowing the cap to be screwed into an implant having a corresponding screw thread. In such embodiments, when a cap of a bone screw assembly is fully seated in a hole of a corresponding implant, the X-axis of the bone-screw assembly is parallel to a long axis of the implant (and of a fractured bone that is being set) In such a manner, the relative motion of the bone fragments fixed to the plate are primarily or exclusively in the X-axis (axial stretching/contraction).

In some embodiments, the maximal extent of the translation in a direction parallel to the X-axis is not less than 0.2 mm, and in some embodiments not less than 0.3 mm.

In some embodiments, the maximal extent of the translation in a direction parallel to the X-axis is not more than 1 mm, not more than 0.9 mm, not more than 0.8 mm and in some embodiments not more than 0.7 mm.

In some embodiments, the limited movement of the screw head within the cavity is predominantly the translation of the screw head in the direction parallel to the X-axis of the cap. In some such embodiments, any movement of the screw head inside the cavity in a direction parallel to the Y-axis of the cap is limited to not more than 0.2 mm, and in some embodiments not more that 0.1 mm.

In some such embodiments, deviation of the screw axis from parallel with the Z-axis is limited to not more than ±20°, and in some embodiments is limited to not more that ±10° or not more than ±5°.

In some embodiments, the limited movement of the screw head within the cavity includes tilting of the screw axis head relative to the Z-axis. In some such embodiments, the screw axis is capable of tilting relative to the Z-axis by up to ±20°, by up ±30° or up to ±45°.

In some embodiments, the tilting is about a single axis. In some such embodiments, the tilting is exclusively around the Y-axis so that during such tilting the screw-shaft rotates in the X-Z plane.

In some embodiments, the tilting is about both the X and Y axes: the X-axis so that during the tilting the screw-shaft rotates in the Y-Z plane; and the Y-axis so that during the tilting the screw-shaft rotates in the X-Z plane.

In some embodiments, a shaft driving-feature is provided on the proximal end of the screw head, to permit torque to be applied to the screw head by a driving tool engaging the shaft driving-feature and thereby enabling the screw to be driven into the bone, and wherein the cap further comprises a proximal port through the top side thereof, which proximal port is dimensioned to allow a driving tool to engage the shaft driving-feature on the proximal end of the screw head.

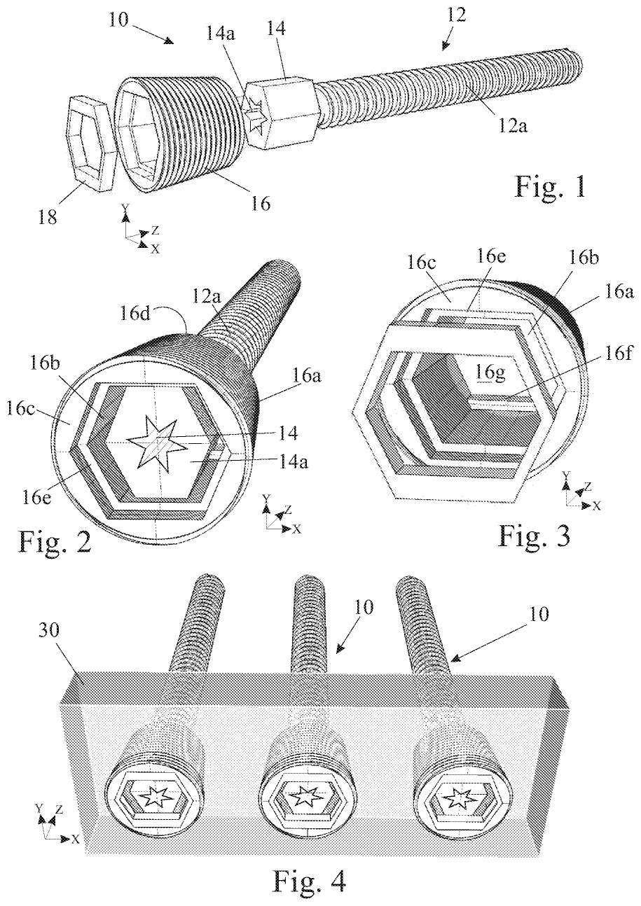

In some such embodiments, the cap and the screw head are coupled for rotation with one another, so that rotation of the screw-shaft by means of the shaft driving-feature on the proximal end of the screw head results in concurrent rotation of the cap. For example, in some such embodiments, the screw head has straight sides parallel to the X-axis and a screw head width defined as the distance between the two straight sides perpendicular to the X-axis, and the cavity has corresponding straight sides parallel to the X-axis and a cavity width defined as the distance between the two straight sides perpendicular to the X-axis, where the screw head width and the cavity width are sufficiently similar so that the fit of the screw head in the cavity is sufficiently tight so that rotation of the screw head around the screw axis leads to rotation of the cap. Such an embodiment is depicted, inter alia, in FIGS. 1, 2 and 3, vide infra.

In some such embodiments, the cap and the screw-shaft are independently rotatable so that rotation of the screw-shaft by means of the shaft driving-feature on the proximal end of the screw head does not lead to concurrent rotation of the cap. For example, in some such embodiments, the screw head is cylindrical and coaxial with the screw-shaft so it can rotate inside the cavity of the cap. In some such embodiments, the height of the screw head and the height of the cavity are sufficiently similar so that the fit of the screw head in the cavity is sufficiently tight to prevent substantial tilting motion.

In some embodiments, the shaft driving-feature on the proximal end of the screw head is an integrally-formed part of the screw head. In some embodiments, the shaft driving-feature on the proximal end of the screw head is part of a separately-formed component fixedly-secured to the screw-shaft.

In some embodiments, the shaft driving-feature on the proximal end of the screw head protrudes from the screw head. In some embodiments, the shaft driving-feature on the proximal end of the screw head is recessed in the screw head.

In some embodiments, the cap further comprises a cap driving-feature capable of being physically engaged by a driving tool to rotate the cap about the Z-axis. In other words, in some embodiments a cap driving-feature is provided on the cap, to permit torque to be applied to the cap by a driving tool engaging the cap driving-feature. In some such embodiments, the cap and the screw head are coupled for rotation with one another, so that rotation of the cap through the cap driving-feature results in concurrent rotation of the screw-shaft. In some such embodiments, the cap is closed (except for the distal port) so that the screw head is inaccessible.

In some embodiments, the screw-shaft has a circular cross section.

In some embodiments, the screw-shaft is substantially cylindrical.

In some embodiments, the shaft screw-thread is self-tapping.

In some embodiments, the screw-shaft is self-drilling.

In some embodiments, the shaft screw-thread is present along the entire length of the screw-shaft, from the distal tip of the screw-shaft to the screw head. Some such embodiments of fully-threaded screws are suitable for use as cortical screws.

In some embodiments, the shaft screw-thread is present only near a distal end of the screw-shaft, there being a portion of the screw-shaft between the shaft screw-thread and the screw head that is devoid of the shaft screw. Some such embodiments are suitable for use as cancellous screws.

In some embodiments, the screw-shaft has a greatest diameter of not less than 1 mm, not less than 2 mm and even not less than 3 mm. In some embodiments, the screw-shaft has a greatest diameter of not more than 10 mm, not more than 9 mm, not more than 8 mm and even not more than 7 mm. In some embodiments, the screw-shaft has a greatest diameter of between 1 mm and 10 mm, and in some embodiments of between 3 mm and 7 mm. As used herein, the term “greatest diameter” relates to the greatest width of the screw shaft (i.e., dimension measured perpendicular to the shaft axis).

In some embodiments, the screw-shaft has a length of not less than 10 mm. In some embodiments, the screw-shaft has a length of not more than 150 mm. In some embodiments, the screw-shaft has a length of between 10 mm and 150 mm.

In some embodiments, the screw-shaft is made of a bio-compatible metal.

In some embodiments, the length and the width of the cap are not less than 2 mm. In some embodiments, the length and width of the cap are not more than 15 mm, not more than 12 mm and even not more than 10 mm. In some embodiments, the length and the width of the cap are between 2 mm and 15 mm, and in some embodiments between 2 mm and 10 mm.

In some embodiments, the cap is made of a bio-compatible metal.

In some embodiments, when in use, the bone screw assembly further comprises an elastic material at least partially filling a volume of the cavity around the screw head. In some embodiments, such an elastic material is a biocompatible elastomer, e.g., a silicone rubber (such as but not limited to Silastic, a Dow Corning Elastomer), a polyether, a polyester urethane, a polyether polyester copolymer, and polypropylene oxide. In some embodiments, the bone screw assembly is provided for use with the elastic material already at least partially filling a volume of the cavity. In some embodiments, the elastic material is introduced into the volume of the cavity, e.g., after the screw-shaft is driven into bone the elastic material is introduced into the volume of the cavity (for example, by a surgeon).

In some embodiments, the cap is formed of separable parts and removal of the screw head from the cavity of the cap is only possible following at least partial separation of the parts.

According to an aspect of some embodiments of the invention there is also provided a kit comprising:

at least one bone screw assembly according to the teachings herein; and

an implant having the form of a bone fixation plate including at least one hole dimensioned to accept the cap of the at least one bone screw assembly.

In some embodiments, the implant is a bone fixation plate as known in the art of bone fracture repair.

In some embodiments, the dimensioning of the at least one hole of the implant is such that when the cap of the at least one bone screw is accepted within the hole, the cap does not protrude beyond a surface of the bone fixation plate.

In some embodiments, the dimensioning of the at least one hole of the implant is such that when the cap of the at least one bone screw is accepted within the hole, the cap cannot tilt relative to the at least one hole.

In some embodiments, the at least one hole of the implant and the cap of the at least one bone screw are substantially truncated cones.

In some embodiments, the at least one hole of the implant and the cap of the at least one bone screw are substantially cylindrical.

In some embodiments, the at least one hole of the implant and the cap of the at least one bone screw include matching threads, allowing the cap to be screwed into the bone fixation plate.

In some embodiments of the kit, the caps of the at least one bone screw assembly and the at least one hole of the implant are shaped and dimensioned so is such that when a cap of a bone screw assembly is fully seated in a hole of the implant, the X-axis of the bone-screw assembly is parallel to a long axis of the implant (and of a fractured bone that is being set) In such a manner, the relative motion of the bone fragments fixed to the plate are primarily or exclusively in the X-axis (axial stretching/contraction).

### BRIEF DESCRIPTION OF THE FIGURES

Some embodiments of the invention are described herein with reference to the accompanying figures. The description, together with the figures, makes apparent to a person having ordinary skill in the art how some embodiments of the invention may be practiced. The figures are for the purpose of illustrative discussion and no attempt is made to show structural details of an embodiment in more detail than is necessary for a fundamental understanding of the invention. For the sake of clarity, some objects depicted in the figures are not to scale.

### In the Figures:

FIG. 1 is an exploded view of an embodiment of a bone screw assembly according to the teachings herein;

FIG. 2 shows an end view of the bone screw assembly of FIG. 1 with a retaining ring omitted;

FIG. 3 is an exploded perspective view of the bone screw assembly of FIG. 1 with the bone screw omitted;

FIG. 4 shows a bone fixation plate receiving three bone screw assemblies as shown in FIGS. 1 to 3: it can be seen that when the cap is fully threaded in the plate, the X-axis of the bone screw assemblies is parallel to the long axis of the plate. This allows the bone fragments and the screw axes fixed thereto to move parallel to the long axis of the plate (the X-axis);

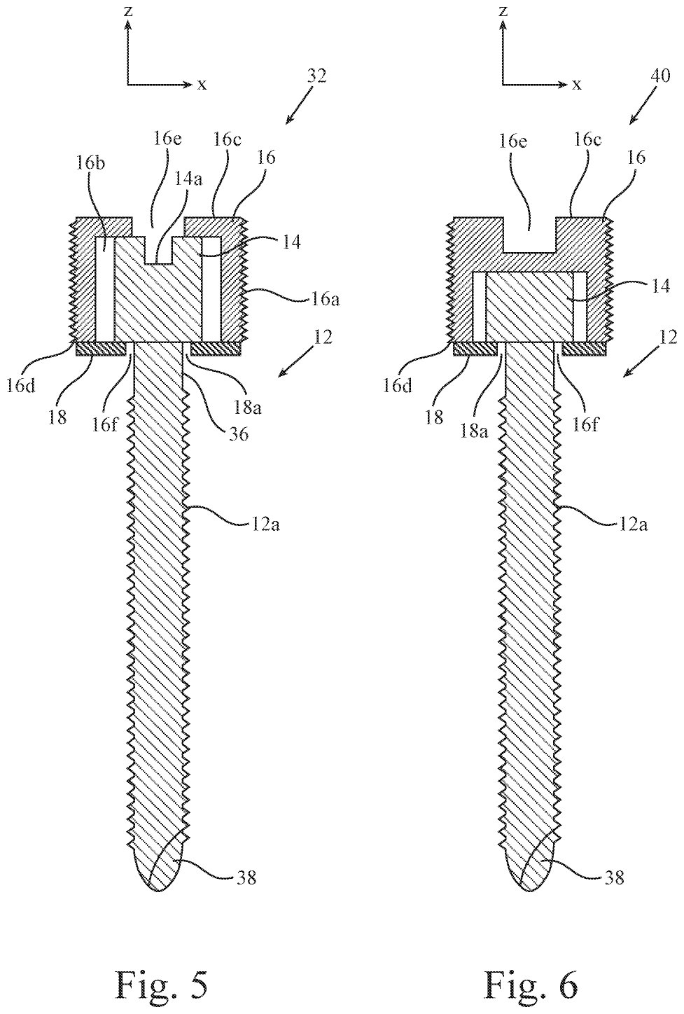

FIG. 5 is a further embodiment of a bone screw assembly according to the teachings herein in side cross section in the X-Z plane;

FIG. 6 is a further embodiment of a bone screw assembly according to the teachings herein in side cross section in the X-Z plane; and

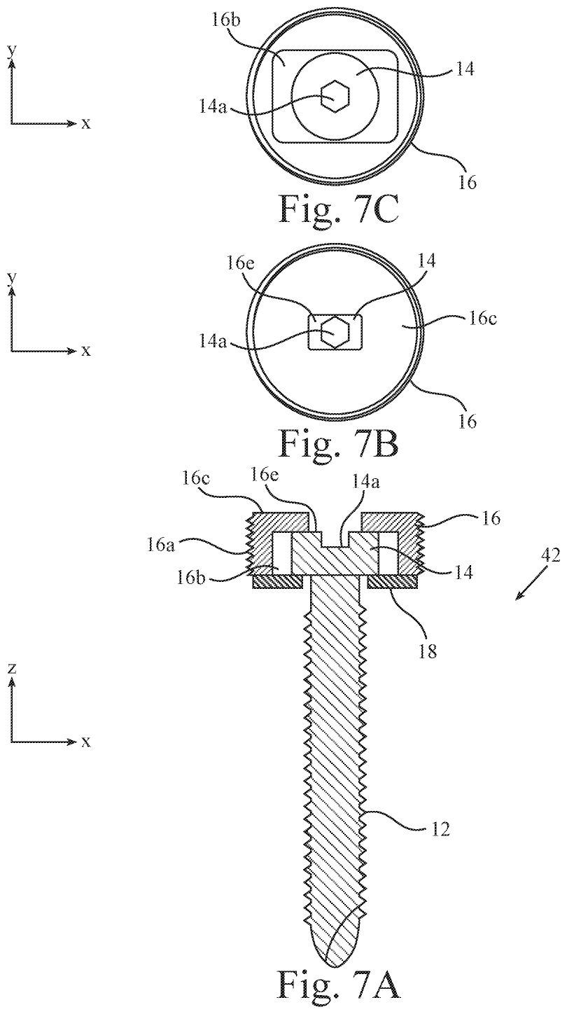

FIGS. 7A, 7B and 7C depict a further embodiment of a bone screw assembly according to the teachings herein: FIG. 7A in side cross section in the X-Z plane, FIG. 7B in top view and FIG. 7C in top cross section in the X-Y plane.

### DESCRIPTION OF SOME EMBODIMENTS OF THE INVENTION

Some embodiments of the invention relate to bone screws and to methods for anchoring bone parts using a bone screw, for example, in order to fix parts of a broken bone in a desired relative orientation.

Before explaining at least one embodiment in detail, it is to be understood that the invention is not necessarily limited in its application to the details of construction and the arrangement of the components and/or methods set forth herein. The invention is capable of other embodiments or of being practiced or carried out in various ways. The phraseology and terminology employed herein are for descriptive purpose and should not be regarded as limiting.

FIGS. 1 to 3 show a bone screw assembly 10 formed of three separate components, namely a bone screw 12 having an enlarged head 14, a cap 16 and a retaining ring 18. All three components are made of a bio-compatible metal, such as stainless steel or titanium. While, in some embodiments, the bone screw 12 and the cap 16 may be separable from one another, in the illustrated embodiment, even prior to insertion of the assembly 10 into a bone, as well as during and after insertion, the three components of the assembly are not separable from one another, the head 14 of the screw 12 being held captive within the cap 16. For example, in some such embodiments, retaining ring 18 is welded to cap 16 to prevent separation of retaining ring 18 from cap 16 and consequent potential separation of bone screw 12 from cap 16.

The bone screw 12 has a screw-threaded cylindrical shaft 12a intended to be driven into a bone. The shaft 12a may be of circular, or other suitable, cross section and is of generally uniform cross section over its length. The screw thread on the outer surface of the shaft 12a may be self-tapping and may extend over the entire length of the shaft 12a. Alternatively, the thread may extend only along the distal end of the shaft 12a, leaving an unthreaded shank at the proximal end, adjacent the head 14. The tip of the shaft 12a may be formed with a cutting edge, similar to the tip of a drill bit, so as to create its own bore in the bone, or it may be designed to engage in a pre-drilled hole in the bone. The screw-shaft 12a may typically have a greatest diameter of between 1 mm and 10 mm, or between 3 mm and 7 mm and its length may be between 10 mm and 150 mm.

The head 14 of the bone screw 12 has a hexagonal cross section that is uniform over its length and its proximal end face is formed with a non-circular recess 14a as a shaft driving-feature for engagement by a driving tool, to enable torque to be applied to the bone screw 12. The recess 14a is illustrated as being intended to receive a Torx® key, but it may be a hexagonal to receive an Allen key, or it may be any suitable driving feature configured to be engaged by an appropriate driving tool and, when the engaged tool is rotated, leading to rotation of screw 12. Furthermore, the driving feature need not be a recess but may be a protrusion that may either be formed as an integral part with the screw-shaft 12a or it may be a part formed separately and secured to the screw-shaft 12a. As a further possibility, the hexagonal head may have no driving feature and torque may be applied to the screw-shaft 12a by means of the cap 16. A driving feature of an assembly according to the teachings herein has any suitable shape allowing engagement with an appropriate driving tool. In some embodiments, the driving feature has a non-circular cross section.

The illustrated cap 16 is formed as truncated cone with a threaded frustoconical outer surface 16a. The cap 16 has an internal cavity 16b of hexagonal cross section (in the X-Y plane) that receives the head 14 of the screw 12. The cap 16 has a top side 16c, a bottom side 16d, a width along an X-axis, a length along a Y-axis and a height along a Z-axis, the three orthogonal axes being shown in FIGS. 1 and 2. The Z-axis that passes through the top side and the bottom side and is nominally parallel to the axis of the screw-shaft 12a.

Though the internal cavity (e.g., internal cavity 16b in FIGS. 1, 2 and 3) may be formed as a blind bore in the bottom side 16d of the cap, it is formed, in the illustrated embodiment, as a through bore with open ports in both the top side 16c and the bottom side 16d of the cap 16. The head 14 of the screw 12 is located within the cap 16 and held captive therein. This is achieved in that the port 16g on the bottom side 16d of the cap is smaller than the head 14 but large enough to allow the shaft screw 12a to pass through it. FIG. 3 shows a ledge 16f formed at the bottom end of the cavity 16b to prevent the head 14 from passing out of the cap 16 through the port 16g.

At the top, or proximal, side of the cap 16, the cavity 16b is formed with a shoulder 16e for receiving an optional hexagonal retaining ring 18. The hole in the ring 18 is large enough to allow a driving tool to connect with the head 14 but small enough to prevent passage of the screw head 14, so that the latter is held captive in the cap 14. The retaining ring 18 is fitted to the cap 16 after the screw head 14 has been located in the cavity 16b and may be held in place by welding, or by the use of an adhesive, or by the use of a force fit or a shrink fit between the retaining ring 18 and the cap 16. The retaining ring 18 serves to hold the head 14 captive in the cap 16 prior to insertion of the bone screw assembly into a bone and may be omitted if the cap 16 and the screw 12 are initially separate components.

As can best be seen in FIG. 2, the head 14 is not dimensioned to be a close fit in the cavity 16b. Instead, the head 14 can move relative to the cap 16 by a small amount, at least in the direction of the X-axis. Thus, the cavity 16b is wider than the head 14 in the X-direction by not less than 0.2 mm or not less than 0.3 mm but does not exceed the width of the head in the X-direction by more than 1 mm or more than 0.9 mm, or more than 0.8 mm or more than 0.7 mm.

In the Y-direction, the head 14 is a close fit in the cavity 16b so that the movement of the screw head within the cap 16 is predominantly parallel to the X-axis of the cap 16. Any movement of the screw head 14 inside the cavity 16b in a direction parallel to the Y-axis of the cap 16 is limited to not more than 0.2 mm or not more than 0.1 mm.

The desired difference in the width of the screw head 14 and the cavity 16b in the X-direction can be achieved by forming the head as a regular hexagon and the cavity 16b as an irregular hexagon that is larger in the X-direction than in the Y-direction. Alternatively, the cavity 16b may be a regular hexagon and the screw head may, as shown, be a flattened hexagon that is narrowed in the X-direction.

On account of the play present in the X-direction, the screw 12 is capable tilting relative to the cap 16 so that its axis does not align with the Z-axis of the cap 16 in the X-Z plane; the tilting taking place about the Y-axis. The deviation of the screw axis from parallel with the Z-axis may be limited to not more than ±20° or not more that ±10° or of not more than ±5°, depending on the length of the head 14 in the direction of the Z-axis. If it is desired to enable the screw to be inserted into the bone at an angle, then the screw axis may be capable of tilting relative to the Z-axis by up to ±20° or up to ±30° or up to ±45°.

The tilting need only be about a single axis, that is to say the screw may only be allowed to tilt in the X-Z plane about the Y-axis. However, if desired, the screw may also be allowed to tilt in the Y-Z plane about the axis. This can be achieved in various ways, for example, by permitting a limited movement of the screw head 14 relative to the cap 16 in the direction of the Y-axis or by tapering the screw head 14 so that its larger proximal end restricts movement in the direction of the Y-axis while its smaller distal end permits pivoting about the X-axis. The same effect may be achieved by forming the cavity 16 such that it tapers in the Y-Z plane, being wider at the proximal end than the distal end.

The bone screw assembly 10 of FIGS. 1 to 3, is used to attach an implant, such as represented by a plate 30 in FIG. 4, to hold together two fragments of a bone that has been fractured to enable the fracture to heal. If the fractured bone is an elongate limb, such as the femur of a leg, the axis of the bone, and the longer axis of the implant plate, will be aligned nominally with the X-axis of the bone screw assembly and the fracture plane would lie nominally in the Y-Z plane. The design of some embodiments of the bone screw assembly according to the teachings herein is such that after the implant plate has been secured to the bone, it prevents movement of the bone parts relative to one another in the Y-Z plane but allows a limited degree of relative movement in the direction of the bone axis, that is to say a relative movement that increases or decreases the gap between the bone parts.

The implant plate 30 is pre-formed with threaded conically tapering cavities to engage with the screw threaded outer surface 16a of the cap 16. Because of the taper, the two threads do not start to engage with one another until the screw threaded shaft 12a has been nearly fully driven into the bone. The bone may be pre-drilled to receive the screw 12 or the tip screw may be designed create a bore as it is driven into the bone. Similarly, a female thread may be pre-formed in the bore in the bone, or the thread on the screw-shaft 12a may be self tapping.

To engage the screw 12 in the bone, torque is applied to the head 14 using the driving feature 14a for example with a driving tool (e.g., Torx® key) engaging the shaft driving-feature 14a. In the case of the illustrated embodiment, the close fit of the screw head 14 in the cavity 16b of the cap in the direction of the Y-axis prevents the screw 12 from rotating relative to the cap, so that torque applied to the driving feature 14a serves also to rotate the cap 16 relative to the implant. In embodiments where the screw 12 and the cap 16 are fast in rotation with one another, as is the case in the illustrated embodiment, the driving feature required to apply torque to the bone screw assembly 10 may be provided on the cap 16 rather than on the head 14 of the screw 12, for example with a driving tool (e.g., Allen key) engaging the hexagonal opening in retaining ring 18 that functions as a cap driving-feature.

The screw 12 is generally driven into the bone so its Z-axis is orthogonal to the plane of the bone fixation plate but on some occasions it may be desired for surgical reasons for screw 12 to be driven into the bone at an angle relative to orthogonal to the plane of the bone fixation plate. The coupling between the head 14 and the cavity 16b will inherently permit a limited degree of tilting of the screw axis in the X-Z plane and, as earlier described, the fit of the head 14 within the cavity 16b may be further designed to permit some tilting in the Y-Z plane.

Towards the end of the penetration of the screw into the bone, the cap will engage in the hole in the implant be screwed firmly into it.

In some embodiments, the lead of the cap/implant hole threads and the lead of the screw-shaft threads are the same. As used herein, the term “lead” as used with reference to a screw has the ordinary meaning with which a person having ordinary skill in the art is familiar, i.e., the distance along screw axis covered by a single 360° rotation.

In some such embodiments, the outer surface of the cap has a single-start threadform having the same pitch as the thread on the screw-shaft so that during implantation when the bone screw assembly is driven into bone and the cap is screwed into the hole in the implant, for a given rotation of the bone screw assembly the axial movement of the cap relative to the plate and the axial movement of the screw-shaft in bone are substantially identical In other such embodiments, the outer surface of the cap has a double-start threadform having half the pitch as the thread on the screw-shaft so that during implantation when the bone screw assembly is driven into bone and the cap is screwed into the the hole in the implant, for a given rotation of the bone screw assembly the axial movement of the cap relative to the plate and the axial movement of the screw-shaft in bone are substantially identical.

In some embodiments, the thread on the outer surface of the cap has a lead that is different from the lead of the thread on the screw-shaft so that during implantation when the bone screw assembly is driven into bone and the cap is screwed into the the hole in the implant, for a given rotation of the bone screw assembly the axial movement of the cap relative to the plate and the axial movement of the screw-shaft in bone are different. In some such embodiments, the lead of the thread on the screw-shaft is smaller (e.g., finer threads) than the lead of the thread on the outer surface of the cap, so that for a given rotation of the bone screw assembly the axial movement of the screw-shaft in bone is less than the axial movement of the cap 16 relative to the plate. Alternatively, in some such embodiments, the lead of the thread on the screw-shaft is larger (e.g., coarser threads) than the lead of the thread on the outer surface of the cap, so that for a given rotation of the bone screw assembly the axial movement of the screw-shaft in bone exceeds the axial movement of the cap 16 relative to the plate, in some such embodiments helping ensure that during use the distal end of the screw head 14 engages the bottom end of the cap 16.

In embodiments where the screw 12 and the cap 16 are not fast in rotation with one another, for example if the head 14 is cylindrical, separate driving features on the head 14 and the cap 16 may be used to ensure that head 14 of the screw engages the bottom surface of the cavity 16b to prevent relative movement in the direction of the Z-axis. In some instances when implanting such embodiments the screw is first driven into the bone using a tool that engages the driving feature in the head of the screw, and when necessary the cap is rotated to engage the implant using a tool that engages the driving feature of the cap.

The dimensioning of the hole in plate 30 may be such that the cap does not protrude beyond a surface of the bone fixation plate. After insertion of the bone screw assembly, it is desirable to seal off the cavity 16b. In order to do so without preventing the desired play in the X-direction between the screw 12 and the implant 30, the cavity may be filled an elastic material.

In the illustrated embodiment, the cap 16 of the bone screw assembly 10 cannot tilt relative to the hole in the implant plate 30. It is however alternatively possible for the cap to be received in an expandable ball of a ball and socket joint, as described for example in FIGS. 4 to 7 of US2007/0233116. Furthermore, while the outer surface of the cap 16 and the hole in the plate 30 have been described as being frustoconical, they may alternatively be cylindrical, vide infra.

An additional embodiment of a bone screw assembly according to the teachings herein, 32 is depicted in side cross section in FIG. 5. In many aspects and details, assembly 32 is similar or identical to bone screw assembly 10 discussed above. Some of the differences between assemblies 10 and 32 are discussed below.

Bone screw assembly 32 consists of three separate components, a bone screw 12 having an enlarged head 14, a cap 16 and a retaining ring 18, all three components made of surgical stainless steel.

Bone screw 12 of assembly 32 has a screw-threaded cylindrical shaft 12a intended to be driven into bone. The screw thread on the outer surface of shaft 12a is self-tapping and extends only along the distal end of the shaft 12a, leaving an unthreaded shank portion 36 at the proximal end of shaft 12a adjacent to head 14. Bone screw 12 is configured as a self-drilling screw, having a distal tip 38 of shaft 12a is formed with a cutting edge so as to create a bore in a bone into which bone screw 12 is driven.

A head 14 of bone screw 12 of assembly 32 has a flattened hexagonal cross section (in the X-Y plane, similar to the depicted in FIG. 2) that is uniform over its length and its proximal end face is formed with a non-circular recess 14a as a shaft driving-feature for engagement by a driving tool, to enable torque to be applied to the bone screw 12.

Cap 16 of assembly 32 is formed as cylinder with a threaded cylindrical outer surface 16a. Cap 16 has an internal cavity 16b of hexagonal cross section in the X-Y plane that receives head 14 of screw 12. Cap 16 has a top side 16c, a bottom side 16d, a width along an X-axis, a length along a Y-axis and a height along a Z-axis, the three orthogonal axes being shown in FIG. 5. The Z-axis that passes through top side 16c and bottom side 16d is nominally parallel to the axis of screw-shaft 12a.

Internal cavity 16b is formed as a through bore with open port 16e in top side 16c and open port 16f in bottom side 16d of cap 16. Head 14 of screw 12 is located within internal cavity 16b of cap 16 and held captive therein. This is achieved in that port 16e on top side 16c of cap 16 is smaller than head 14 so that head 14 cannot pass through port 16e. In contrast, port 16f on bottom side 16d is large enough so that head 14 can pass therethrough, allowing head 14 of screw 12 to pass through port 16f into cavity 16b of cap 16. To prevent screw 12 from separating from cap 16, e.g., by head 14 falling out of cavity 16b through port 16f, retaining ring 18 is secured (e.g., by welding) to bottom side 16d of cap 16.

Retaining ring 18 is a flat stainless steel ring which dimensions and shape in the X-Y plane are identical to those of bottom side 16d of hollow cap 16, although in some similar embodiments the retaining ring is smaller than the bottom side of the respective cap. A hole 18a of retaining ring 18 is configured to prevent passage of screw head 14 therethrough but allows motion of screw shaft 12a in the X-Y plane, specifically, in the X-Y plane of hole 18a is smaller than screw head 14 but larger than screw shaft 12a. Retaining ring 18 is welded to bottom side 16a of cap 16 so that screw head is contained (held captive) inside cavity 16b of cap 16.

Port 16e on top side 16c of cap 16 has a non-circular shape and is dimensioned to accept the distal end of a driving tool. Accordingly, port 16e is configured to function as a cap driving-feature of assembly 32 for engagement by a driving tool, to enable torque to be applied to cap 16 and, through cap 16, to bone screw 12. Specifically, when an appropriate driving tool is engaged with port 16e and rotated, the driving tool applies torque to the inner surfaces of port 16e, thereby rotating cap 16. Since cavity 16b has a hexagonal cross section and since screw head 14 has a hexagonal cross section that snugly fits inside cavity 16b (similar or identical to the depicted in FIG. 2), the inside surfaces of cavity 16b apply torque to screw head 14, thereby rotating screw 12. In some embodiments of an assembly according to the teachings herein that are similar or identical to assembly 32 depicted in FIG. 5, a port in a top side of a cap (analogous to port 16e of assembly 32) is not configured as a cap driving-feature, e.g., is round. In such embodiments, torque to drive the screw and the cap is applied through a shaft driving-feature in a head of the screw (analogous to recess 14a of assembly 32).

The relative shape and dimensions of screw head 14 and cavity 16b of cap 16 are such that there is substantially no tilting of screw 12 relative to cap 16 possible. Further, screw head 14 and cap 16 are rotationally coupled so when one is rotated by application of torque through a respective shaft-driving/cap driving-feature, the other also rotates. However, as depicted in FIG. 5, in the X-dimension screw head 14 is substantially smaller than cavity 16b and screw shaft 12a is substantially smaller than hole 18a in retaining ring 18, thereby allowing a limited movement of screw head 14 within cavity 16b, the limited movement including translation of screw head 14 and screw shaft 12a in a direction parallel to the X-axis of cap 16 in the X-Z plane.

An additional embodiment of a bone screw assembly according to the teachings herein, 40 is depicted in side cross section in FIG. 6. In many aspects and details, assembly 40 is similar or identical to bone screw assembly 32 discussed above. The differences between assemblies 40 and 32 are discussed below.

As seen in FIG. 6, cap 16 of assembly 40 is formed as a blind bore in a bottom side 16d of cap 16 accessible through hole 18a of retaining ring 18 and port 16f.

Screw head 14 of screw 12 is inaccessible and devoid of a shaft driving-feature.

In assembly 40, cap 16 includes a non-circular (in the X-Y plane) recess 16e on a top side 16c as a cap driving-feature for engagement by a driving tool, to enable torque to be applied to cap 16 and, through cap 16, to bone screw 12. The use and functioning of recess 16e as a cap driving-feature is substantially the same as described above for assembly 32 depicted in FIG. 5.

As discussed for assembly 32, in assembly 40, the relative shape and dimensions of screw head 14 and cavity 16b of cap 16 are such that there is substantially no tilting of screw 12 relative to cap 16 possible. Further, screw head 14 and cap 16 are rotationally coupled so when cap 16 is rotated by application of torque through recess 16e (the cap driving-feature of assembly 32), screw 12 rotates. As discussed for assembly 32, in the X-dimension screw head 14 is substantially smaller than cavity 16b and screw shaft 12a is substantially smaller than hole 18a in retaining ring 18, thereby allowing a limited movement of screw head 14 within cavity 16b, the limited movement including translation of screw head 14 and screw shaft 12a in a direction parallel to the X-axis of cap 16 in the X-Z plane.

An additional embodiment of a bone screw assembly according to the teachings herein, 42 is depicted in FIGS. 7: FIG. 7A in side cross section in the X-Z plane, FIG. 7B in top view in the X-Y plane and FIG. 7C in top cross section in the X-Y plane.

Assembly 42 is identical with assembly 32 depicted in FIG. 5 with a few differences discussed hereinbelow.

Bone screw assembly 42 consists of three separate components, a bone screw 12 having an enlarged head 14, a cap 16 and a retaining ring 18, all three components made of surgical stainless steel.

Bone screw 12 of assembly 42 is identical to that of assembly 32 except for screw head 14. Unlike screw head 14 of assembly 32 which has a flattened hexagonal cross section in the X-Y plane (similar to the depicted in FIG. 2), a screw head 14 of assembly 42 has a circular cross section in the X-Y plane as is seen in FIG. 7C. On the proximal (top) face of screw head 14 is a hexagonal recess 14a as a shaft driving-feature for engagement by a driving tool, to enable torque to be applied to bone screw 12 of assembly 42, leading to rotation thereof. Importantly, since screw head 14 has circular cross section, there is no rotational coupling with cap 16 of assembly 42 so rotation of screw 12 through recess 14a does not lead to rotation of cap 16.

As in assembly 32, cap 16 of assembly 42 is formed as cylinder with a threaded cylindrical outer surface 16a. Cap 16 has an internal cavity 16b of rounded-rectangle cross section in the X-Y plane (see FIG. 7C) that receives head 14 of screw 12.

Internal cavity 16b of cap 16 is formed as a through bore with an open port 16e in a a top side 16c of cap 16. Port 16e on top side 16c of cap 16 is smaller than screw head 14 so that head 14 cannot pass through port 16e. However, port 16e is dimensioned to allow an appropriate driving tool to access hexagonal recess 14a of screw head 14. Port 16e has a rounded-rectangle shape in the X-Y plane (see FIG. 7B) and is dimensioned to accept the distal end of a driving tool such as a screw driver. Accordingly, port 16e is configured to function as a cap driving-feature of assembly 42 for engagement by a driving tool, to enable torque to be applied to cap 16.

During use of assembly 42, a user uses a first appropriate driving tool to engage recess 14a in screw head 14 through port 16e and then to rotate screw 12 using the first driving tool to drive screw 12 through a hole in an implant into a bone until retaining ring 18 contacts the implant. The user changes to a second driving tool to engage port 16e in cap 16 and then rotate cap 16 using the second driving tool to drive cap 16 into the hole in the implant while screw 12 does not rotate. As cap 16 is driven downwards, screw 12 does not move until the proximal face of screw head 14 contacts the upper surface of cavity 16b. The user then alternates between using the first tool to drive screw 12 into the bone and using the second tool to drive cap 16 into the hole in the implant until a desired depth of penetration into the bone is achieved. In some embodiments, instead of two different driving tools, a single driving tool that can simultaneously engage both recess 14a in screw head 14 and port 16e in cap 16 is used.

Bone screw assemblies 32, 40 and 42 are optionally provided as components of a kit, substantially as described for bone screw assembly 10.

Bone screw assemblies 32, 40 and 42 are typically used in a manner to attach an implant such as a plate, substantially as described for bone screw assembly 10 and depicted in FIG. 4.

Unless otherwise defined, all technical and scientific terms used herein have the same meaning as commonly understood by one of ordinary skill in the art to which the invention pertains. In case of conflict, the specification, including definitions, will take precedence.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

As used herein, the indefinite articles “a” and “an” mean “at least one” or “one or more” unless the context clearly dictates otherwise.

As used herein, when a numerical value is preceded by the term “about”, the term “about” is intended to indicate +/−10%.

As used herein, a phrase in the form “A and/or B” means a selection from the group consisting of (A), (B) or (A and B). As used herein, a phrase in the form “at least one of A, B and C” means a selection from the group consisting of (A), (B), (C), (A and B), (A and C), (B and C) or (A and B and C).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and scope of the appended claims.

Citation or identification of any reference in this application shall not be construed as an admission that such reference is available as prior art to the invention.

1. A bone screw assembly for securing an implant to a bone comprising:
(i) a screw-shaft bearing a shaft screw-thread, said screw-shaft defining a screw axis and having a distal tip and a proximal end, said screw-shaft, said shaft screw-thread and said distal tip configured to penetrate and engage the bone;
(ii) an enlarged screw head located at said proximal end of said screw-shaft;
(iii) a cap for engaging the implant having a top side, a bottom side, a width along an X-axis, a length along a Y-axis and a height along a Z-axis that passes through said top side and said bottom side, said cap having an inner surface defining a cavity in which said screw head is retained, a distal port being provided in the bottom side of the cap through which said screw-shaft passes, said distal port being dimensioned to prevent passage of said screw head therethrough;
wherein said internal cavity, said distal port, said screw-shaft and said screw head are dimensioned to allow, during use, a translation of said screw head in a direction parallel to said X-axis of said cap, while limiting translation of said screw head in a direction parallel to said Y-axis of said cap.

2. The bone screw assembly of claim 1, wherein the maximal extent of said translation in a direction parallel to said X-axis is not less than 0.2 mm.

3. The bone screw assembly of claim 2, wherein the maximal extent of said translation in a direction parallel to said X-axis is not more than 1 mm.

4. (canceled)

5. The bone screw assembly of claim 2, wherein any movement of said screw head inside said cavity in a direction parallel to said Y-axis of said cap is limited to not more than 0.2 mm.

6-11. (canceled)

12. The bone screw assembly of claim 1, wherein a shaft driving-feature is provided on the proximal end of the screw head, to permit torque to be applied to the screw head by a driving tool engaging the shaft driving-feature and thereby enabling the screw to be driven into a bone, and wherein said cap further comprises a proximal port through said top side thereof, which proximal port is dimensioned to allow a driving tool to engage said shaft driving-feature on the proximal end of the screw head.

13. The bone screw assembly of claim 12, wherein said cap and said screw head are coupled for rotation with one another, so that rotation of said screw-shaft by means of said shaft driving-feature results in concurrent rotation of said cap.

14-18. (canceled)

19. The bone screw assembly of claim 1, wherein said cap further comprises a cap driving-feature capable of being physically engaged by a driving tool to rotate said cap about said Z-axis.

20. The bone screw assembly of claim 19, wherein said cap and said screw head are coupled for rotation with one another, so that rotation of said cap through said cap driving-feature results in concurrent rotation of said screw-shaft.

21. The bone screw assembly of claim 1, wherein said cap further comprises a cap screw thread on an outer surface thereof allowing said cap to be screwed into an implant having a corresponding screw thread.

22-26. (canceled)

27. The bone screw assembly of claim 1, wherein said shaft screw-thread is present only near a distal end of said screw-shaft, there being a portion of said screw-shaft between said shaft screw-thread and said screw head that is devoid of said shaft screw.

28-32. (canceled)

33. The bone screw assembly of claim 1, wherein, in use, the assembly further comprises an elastic material at least partially filling a volume of said cavity around said screw head.

34. (canceled)

35. A kit comprising:
at least one bone screw assembly of claim 1; and
an implant having the form of a bone fixation plate including at least one hole dimensioned to accept said cap of said at least one bone screw assembly.

36. The kit of claim 35, wherein the dimensioning of said at least one hole is such that when said cap of said at least one bone screw is accepted within said hole, said cap does not protrude beyond a surface of said bone fixation plate.

37. (canceled)

38. The kit of claim 35, wherein said at least one hole of said implant and said cap of said at least one bone screw are substantially truncated cones.

39. The kit of claim 35, wherein said at least one hole of said implant and said cap of said at least one bone screw are substantially cylindrical.

40. The kit of claim 35, wherein said at least one hole of said implant and said cap of said at least one bone screw include matching threads, allowing said cap to be screwed into said bone fixation plate.

41. The kit of claim 35, the caps of said at least one bone screw assembly and said at least one hole of said implant are shaped and dimensioned so is such that when a said cap of a said bone screw assembly is fully seated in a said hole of said implant, said X-axis of said bone-screw assembly is parallel to a long axis of said implant.

42. The kit of claim 35 wherein when the plate is used in the repair of a bone of a fractured elongate limb by means of the screw fixing said plate to the bone of the fractured elongate limb, so that the cap is locked in the plate, the screw head can move relative to the cap and the plate in along the X-axis, which is the direction parallel to the long axis of the bone.

43. The kit of claim 42 wherein the X-axis is parallel to the long axis of the implant.

44. The bone screw assembly of claim 1, wherein when the implant is secured to a bone using the bone screw assembly, movement of bone parts relative to one another in the Y-Z plane is prevented relative to movement in the direction of the X-axis, wherein the bone axis is parallel to the X-axis.
